(19) United States
(12) Patent Application Publication
(10) Pub. No.: US 2019/0008784 A1
(43) Pub. Date: Jan. 10, 2019

(54) NOVEL SECNIDAZOLE SOFT GELATIN CAPSULE FORMULATIONS AND USES THEREOF

(71) Applicant: SYMBIOMIX THERAPEUTICS, LLC, NEWARK, NJ (US)

(72) Inventors: ROBERT JACKS, HAMDEN, CT (US); LINUS FONKWE, CLEARWATER, FL (US); IRENA MCGUFFY, LUTZ, FL (US)

(73) Assignee: SYMBIOMIX THERAPEUTICS, LLC, NEWARK, NJ (US)

(21) Appl. No.: 16/014,809

(22) Filed: Jun. 21, 2018

Related U.S. Application Data
(60) Provisional application No. 62/529,991, filed on Jul. 7, 2017.

(51) Int. Cl. A61K 9/48; A61K 9/00; A61K 31/4164; A61P 31/04

(52) U.S. Cl. CPC A61K 9/4825; A61K 9/4858; A61P 31/04; A61K 31/4164; A61K 9/0034

(57) ABSTRACT

Embodiments described herein are directed to novel soft gelatin capsule formulations for intravaginal administration comprising secnidazole compounds and methods and uses of these pharmaceutical compositions in the treatment of bacterial vaginosis.

### CROSS-REFERENCE TO RELATED APPLICATIONS

This application claims the benefit of priority in and to U.S. Provisional Application No. 62/529,991, entitled “Novel Secnidazole Soft Gelatin Capsule Formulations and Uses Thereof” filed Jul. 7, 2017, the disclosure of which is incorporated by reference in its entirety.

### TECHNICAL FIELD

The present invention relates to novel soft gelatin capsule formulations for intravaginal administration comprising secnidazole compounds and uses of these pharmaceutical compositions in the treatment of bacterial vaginosis.

All references and products cited within this application are incorporated by reference in their entirety.

### BACKGROUND

Urogenital infections affect more than one billion women per year, all over the world, representing one of the main reasons for women to seek medical consultation. Bacterial vaginosis (BV) is one of the main causes of vaginal problems, is the cause of malodorous discharge in women of reproductive age, and affects an estimated 10-15% of reproductive aged women. It is also associated with several public health problems, including, preterm birth and acquisition and/or transmission of sexually transmitted diseases (Eschenbach D. A., Clin. Infect. Dis. (1993) 16:S282-287; Hillier S., AIDS Res. Hum. Retroviruses (1998) 14:17-21).

BV is a complex disease and occurs due to change in normal vaginal flora. The precise cause of BV, however, it is not yet clear and it is hypothesized that BV may be caused by an increase in the number of Gardnerella vaginalis and other anaerobes in vagina, along with a decrease in lactobacilli (Hill G. B., Am. J. Obstet. Gynecol. (1993) 169:450-4; Ferris et al., J. Clin. Microbiol. (2004) 42:5892-4). Vaginal biopsy sample studies have revealed that BV consists of a dense biofilm in which G. vaginalis is predominant (Swidsinski et al., Obstet. Gynecol. (2005) 106:1013-1023).

Delivering medications, particularly high doses, intravaginally also poses several challenges. The rate and extent of drug absorption after intravaginal administration depends on several factors such as formulation, vaginal physiology, age of the patient and menstrual cycle of the patient. Further, considerable variability in the rate and extent of absorption of vaginally administered drugs is observed by changes in thickness of vaginal epithelium. Issues such as cultural sensitivity, personal hygiene, gender specificity, local irritation and influence of sexual intercourse, also need to be addressed during the design of a vaginal formulation.

Manufacturing high dosage formulations of drugs, such as those used for the treatment of BV, also poses several unique challenges. For instance, making compositions with high dosage of a drug can be complicated by solubility issues due to high concentration of the drug, precipitation of one or more components present in the composition due to high concentration of the drug, difficulties in finding an appropriate composition of excipients that provide suitable viscosity of the final drug composition, or a combination thereof.

Achieving suitable pharmacokinetics when a high dosage of a drug is incorporated in a composition may also be difficult at times. For example, high dosage of a drug may cause an unwanted spike in the serum concentration of the drug at an undesirable time after administration or cause chemical or physical interactions with excipients present in the composition causing delays in delivery of the drug due to, e.g., precipitation of one or more components of the composition.

Accordingly, there is a pending need in the medical and pharmaceutical arts to develop a pharmaceutical composition suitable for treatment of BV in a subject in need thereof, wherein the treatment has better efficacy and tolerability than the currently available treatments. The present invention is directed to overcoming these and other deficiencies in the art.

### SUMMARY OF THE INVENTION

Exemplary embodiments herein are directed to soft gelatin capsule compositions for vaginal administration of secnidazole comprising a soft gelatin capsule and a therapeutically effective amount of secnidazole dispersed in a mono unsaturated fatty acid excipient. Further embodiments are directed to soft gelatin capsule compositions for vaginal administration of secnidazole comprising a soft gelatin capsule and a therapeutically effective amount of secnidazole dispersed in a mono unsaturated fatty acid excipient. Further embodiments include the therapeutically effective amount of secnidazole is from 500 milligrams to about 1,000 milligrams, or about 750 milligrams. Further embodiments include the mono unsaturated fatty acid excipient is a polyoxylglyceride excipient. Further embodiments include the polyoxylglyceride excipient comprises monoglycerides Oleic acid (C18:1), diglycerides Oleic acid (C18:1), triglycerides Oleic acid (C18:1), mono-polyethylene glycol-6 esters of Oleic acid (C18:1), di-polyethylene glycol-6 esters of Oleic acid (C18:1), or any combination thereof. Further embodiments include the mono unsaturated fatty acid excipient is Labrafil M1944 CS, Labrafac Lipophile WL1349, or a combination thereof. Further embodiments include the amount of polyoxylglyceride excipient or mono unsaturated fatty acid excipient is about 1.2 grams.

Other exemplary embodiments herein are directed to methods of treating bacterial vaginosis in a patient in need thereof, comprising administering to the patient a soft gelatin capsule composition comprising a therapeutically effective amount of a nitroimidazole, such as secnidazole, dispersed in a mono unsaturated fatty acid excipient, wherein the soft gelatin capsule composition is administered intravaginally to the patient. Yet other exemplary embodiments are directed to methods of treating bacterial vaginosis in a patient in need thereof comprising administering to the patient a soft gelatin capsule composition comprising a therapeutically effective amount of secnidazole, dispersed in a mono unsaturated fatty acid excipient, wherein the soft gelatin capsule composition is administered intravaginally to the patient. Further embodiments include the soft gelatin capsule composition is administered intravaginally to the patient and said bacterial vaginosis is treated. Further embodiments include the therapeutically effective amount of secnidazole is from 500 milligrams to about 1,000 milligrams, or about 750 milligrams. Further embodiments include the mono unsaturated fatty acid excipient is a polyoxylglyceride excipient. Further embodiments include the polyoxylglyceride excipient comprises monoglycerides Oleic acid (C18:1), diglycerides Oleic acid (C18:1), triglycerides Oleic acid (C18:1), mono-polyethylene glycol-6 esters of Oleic acid (C18:1), di-polyethylene glycol-6 esters of Oleic acid (C18:1), or any combination thereof. Further embodiments include the mono unsaturated fatty acid excipient is Labrafil M1944 CS, Labrafac Lipophile WL1349, or a combination thereof. Further embodiments include the amount of polyoxylglyceride excipient or mono unsaturated fatty acid excipient is about 1.2 grams.

### BRIEF DESCRIPTION OF FIGURE

FIG. 1 depicts a PXRD overlay of secnidazole residual solids after precipitation from binary mixtures.

### DETAILED DESCRIPTION

Current nitroimidazole drugs such as secnidazole are effective in the treatment of several conditions including bacterial vaginosis. In some instances, administration of a nitroimidazole directly to the vagina is preferable due to toxicity related with oral administration in certain patients. Studies have shown that higher doses of the nitroimidazole metronidazole results in a superior therapeutic effect in the treatment of bacterial vaginosis. In one example, Sanchez et al. (American Journal of Obstetrics and Gynecology (2004) 191, 1898-906) demonstrated that 500 mg ovule formulation of metronidazole was significantly more effective than a 37.5 mg metronidazole gel in the treatment of bacterial vaginosis. In another example, Aguin et al. (Journal of Lower Genital Tract Disease, Volume 18, Number 2, 2014, 156-161) demonstrated that intravaginal doses of a 750 mg ovule formulation of metronidazole resulted in a higher cure rates than intravaginal doses of a 500 mg ovule composition of metronidazole. However, these elevated doses of drug are hard to incorporate into a formulation for vaginal administration due to the effect of the large amounts of solid drug on flowability of the excipients used in the manufacture of compositions suitable for vaginal administration. Ovule formulations have been formulated using mineral oils which are capable of carrying high drug loads but the interaction of mineral oil with latex poses problems for patients using contraceptives such as condoms to prevent pregnancy and avoid transmission of sexual transmitted diseases. Vaginal suppositories have been used, patients frequently complain that these formulations are messy and inconvenient to use. Applicant has developed a novel soft gel capsule formulation capable of holding the large doses of secnidazole that are required for the optimal intravaginal treatment of infections such as bacterial vaginosis. The novel soft gel capsule formulations developed by Applicant allow for high doses of secnidazole to be loaded into a single soft gel capsule using an excipient that retains suitable flow properties when mixed with the secnidazole and that is also compatible with latex condoms. The soft gel capsule compositions of the present invention also possess optimal patient handling and administration properties due to the encapsulation of the drug excipient dispersions.

Applicant has developed novel soft gelatin capsule compositions comprising therapeutically effective amounts of secnidazole which can be safely and effectively administered intravaginally.

Before the present formulations and methods are described, it is to be understood that this invention is not limited to the particular processes, compounds, or methodologies described, as these may vary. It is also to be understood that the terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described.

In each of the embodiments disclosed herein, the compounds and methods may be utilized with or on a subject in need of such treatment, which may also be referred to as “in need thereof.” As used herein, the phrase “in need thereof” means that the subject has been identified as having a need for the particular method or treatment and that the treatment has been given to the subject for that particular purpose.

As used herein, the term “patient” and “subject” are interchangeable and may be taken to mean any living organism, which may be treated with compounds of the present invention. As such, the terms “patient” and “subject” may include, but is not limited to, any non-human mammal, primate or human. In some embodiments, the “patient” or “subject” is an adult, child, infant, or fetus. In some embodiments, the “patient” or “subject” is a human. In some embodiments, the “patient” or “subject” is a mammal, such as mice, rats, other rodents, rabbits, dogs, cats, swine, cattle, sheep, horses, primates, or humans.

As used herein, the terms “adjunctive administration” and “adjunctively” may be used interchangeably and refer to simultaneous administration of more than one compound in the same dosage form, simultaneous administration in separate dosage forms, and separate administration of more than one compound as part of a single therapeutic regimen.

It must be noted that, as used herein, and in the appended claims, the singular forms “a”, “an” and “the” include plural reference unless the context clearly dictates otherwise.

As used herein, the term “about” means plus or minus 10% of the numerical value of the number with which it is being used. Therefore, about 50% means in the range of 45%-55%.

“Optional” or “optionally” may be taken to mean that the subsequently described structure, event or circumstance may or may not occur, and that the described includes instances where the event occurs and instances where it does not.

“Administering” when used in conjunction with a therapeutic means to administer a therapeutic directly or indirectly into or onto a target tissue to administer a therapeutic to a patient whereby the therapeutic positively impacts the tissue to which it is targeted. “Administering” a composition may be accomplished by oral administration, injection, infusion, inhalation, absorption or by any method in combination with other known techniques. “Administering” may include the act of self-administration or administration by another person such as a health care provider.

As used here, the term “therapeutic” means an agent utilized to treat, combat, ameliorate or prevent an unwanted disease, condition or disorder of a patient.

The terms “therapeutically effective amount” or “therapeutic dose” as used herein are interchangeable and may refer to the amount of an active agent or pharmaceutical compound or composition that elicits a clinical, biological or medicinal response in a tissue, system, animal, individual or human that is being sought by a researcher, veterinarian, medical doctor or other clinical professional. A clinical, biological or medical response may include, for example, one or more of the following: (1) preventing a disease, condition or disorder in an individual that may be predisposed to the disease, condition or disorder but does not yet experience or display pathology or symptoms of the disease, condition or disorder, (2) inhibiting a disease, condition or disorder in an individual that is experiencing or displaying the pathology or symptoms of the disease, condition or disorder or arresting further development of the pathology and/or symptoms of the disease, condition or disorder, and (3) ameliorating a disease, condition or disorder in an individual that is experiencing or exhibiting the pathology or symptoms of the disease, condition or disorder or reversing the pathology and/or symptoms experienced or exhibited by the individual.

As used herein, the term “daily dose amount” refers to the amount of an active agent per day that is administered or prescribed to a patient. This amount can be administered in multiple unit doses or in a single unit dose, in a single time during the day or at multiple times during the day.

The term “treating” may be taken to mean prophylaxis of a specific disorder, disease or condition, alleviation of the symptoms associated with a specific disorder, disease or condition and/or prevention of the symptoms associated with a specific disorder, disease or condition. In some embodiments, the term refers to slowing the progression of the disorder, disease or condition or alleviating the symptoms associated with the specific disorder, disease or condition. In some embodiments, the term refers to alleviating the symptoms associated with the specific disorder, disease or condition. In some embodiments, the term refers to alleviating the symptoms associated with the specific disorder, disease or condition. In some embodiments, the term refers to restoring function which was impaired or lost due to a specific disorder, disease or condition.

The term “pharmaceutical composition” shall mean a composition including at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan. A pharmaceutical composition may, for example, contain secnidazole or a pharmaceutically acceptable salt of secnidazole as the active ingredient.

“Pharmaceutically acceptable salt” is meant to indicate those salts which are, within the scope of sound medical judgment, suitable for use in contact with the tissues of a patient without undue toxicity, irritation, allergic response and the like, and are commensurate with a reasonable benefit/risk ratio. Pharmaceutically acceptable salts are well known in the art. For example, Berge et al. (1977) J. Pharm. Sciences, Vol 6. 1-19, describes pharmaceutically acceptable salts in detail. A pharmaceutical acceptable “salt” is any acid addition salt, preferably a pharmaceutically acceptable acid addition salt, including, but not limited to, halogenic acid salts such as hydrobromic, hydrochloric, hydrofloric and hydroiodic acid salt; an inorganic acid salt such as, for example, nitric, perchloric, sulfuric and phosphoric acid salt; an organic acid salt such as, for example, sulfonic acid salts (methanesulfonic, trifluoromethan sulfonic, ethanesulfonic, benzenesulfonic or p-toluenesufonic, acetic, malic, fumaric, succinic, citric, benzonic gluconic, lactic, mandelic, mucic, pamoic, pantothenic, oxalic and maleic acid salts; and an amino acid salt such as aspartic or glutamic acid salt. The acid addition salt may be a mono- or di-acid addition salt, such as a di-hydrohalogic, di-sulfuric, di-phosphoric or di-organic acid salt. In all cases, the acid addition salt is used as an achiral reagent which is not selected on the basis of any expected or known preference for the interaction with or precipitation of a specific optical isomer of the products of this disclosure.

As used herein the term “soft gelatin capsule” shall mean a gelatin-based shell surrounding a liquid or solid fill. The soft gelatin capsules for use in the compositions described herein may be made up of a combination of gelatin, water, an opacifier and a plasticizer such as glycerin or sorbitol. The soft gelatin capsule of the present invention will be filled with an effective amount of secnidazole and a mono unsaturated fatty acid excipient whose physical properties, such as shape, size and consistency will facilitate its therapeutic use via intravaginal administration.

As used herein the term “soft gelatin capsule composition” includes the soft gelatin capsule or any suitable encapsulation medium known in the art that is suitable for vaginal administration along with any liquid or solid fill, including the secnidazole compositions of this invention. For example, a soft gelatin capsule composition may comprise a therapeutically effective amount of secnidazole and a mono unsaturated fatty acid excipient along with soft gelatin capsule shell. In some embodiments, the soft gelatin capsule composition contains the therapeutically effective amount of secnidazole and a mono unsaturated fatty acid excipient and, optionally, any suitable encapsulation medium known in the art that is suitable for vaginal administration and will be suitable for dissolution and dispersion of the contents of the encapsulation medium in the vagina but will retain its integrity while being stored prior to use. In some embodiments, the soft gelatin capsule compositions disclosed herein are highly storage stable compositions having long term storage stability while providing efficacy when used to treat the conditions disclosed herein including, but not limited to bacterial vaginosis.

The soft gelatin capsule compositions of the present invention may be produced in a process known as encapsulation using the rotary die encapsulation process. The encapsulation process may be described as a form/fill/seal process. In some embodiments, two flat ribbons of shell material are manufactured on the machine and brought together on a twin set of rotating dies. The dies contain recesses in the desired size and shape, which cut out the ribbons into a two-dimensional shape, and form a seal around the outside. At the same time a pump delivers a precise dose of fill material (i.e. a therapeutically effective amount of secnidazole and a mono unsaturated fatty acid excipient) through a nozzle incorporated into a filling wedge whose tip sits between the two ribbons in between two die pockets at the point of cut out. The wedge is heated to facilitate the sealing process. The wedge injection causes the two flat ribbons to expand into the die pockets, giving rise to the three-dimensional finished product. After encapsulation, the soft gelatin capsule compositions are dried for about two days to about two weeks.

The contents of soft gelatin capsule compositions of the present invention may be solid or liquid at room temperature, and preferably have a flow point in the range of 30 to 40° C.; more preferably 30 to 37° C. The flow point is visually determined based upon heating a sample from 25° C. at a rate of 2° C. per minute and observing the temperature at which rapid flow of the sample occurs. This measurement is conveniently carried out using a microscope equipped with a video camera having on-screen digital monitoring of the temperature. In some embodiments the contents of the soft gelatin capsule compositions may be liquid at room temperature

The total weight of the soft gelatin capsule compositions of the present invention will vary according to the amount of active ingredient and “ease of use” characteristics such as size and shape of the resulting suppository and is therefore not critical. Generally, lower amounts of active ingredient may be accommodated by a smaller size suppository (including, e.g., an ovule or a capsule), and higher amounts of active ingredient will require a larger size suppository. Manufacturing properties, such as the viscosity of the secnidazole base dispersion, when the base is in the molten state during processing, will also determine the minimum amount of suppository base that is needed to disperse, mold and package a suppository having a given amount of secnidazole. Such a parameter is not critical to the present invention and may be determined in the course of routine optimization of the manufacturing process. Typical soft gelatin capsule compositions will be in the range of 0.5 to 10 g, preferably 1 to 5 g, and most preferably 1 to 3 g. Thus, compositions would generally be in the range of 0.1% to 60% secnidazole. Preferably 20% to 40%, more preferably 30% to 40%, and most preferably 35% to 40%. In some embodiments, the soft gelatin capsule compositions will comprise about 38% secnidazole.

The soft gelatin capsule compositions of the present invention may also contain additives, such as stabilizers (e.g., antioxidants and other types of preservatives), polymorphic transition accelerators (e.g., tristearin), biocompatible polymers, surfactants, dispersants, water absorbents and the like. The use of biocompatible polymers, surfactants and water absorbents are described in U.S. Pat. No. 4,765,978, the disclosure of which is hereby incorporated by reference. The concentration of these additives may vary according to the particular additive used and the desired result sought. The use of the kind and concentration of additives are well within the ability of the skilled artisan.

Embodiments described herein are directed to novel soft gelatin capsule composition for vaginal administration of secnidazole to treat a bacterial infection of the vagina such as, but not limited to, bacterial vaginosis in a patient in need thereof.

Some embodiments are directed to soft gelatin capsule compositions for vaginal administration of secnidazole comprising a soft gelatin capsule and a therapeutically effective amount of secnidazole dispersed in a mono unsaturated fatty acid excipient.

In some embodiments, the therapeutically effective amount of secnidazole is from about 500 milligrams to about 1,000 milligrams per capsule. In some embodiments, the therapeutically effective amount of secnidazole is from about 600 milligrams to about 900 milligrams. In some embodiments, the therapeutically effective amount of secnidazole is from about 650 milligrams to about 850 milligrams. In some embodiments, the therapeutically effective amount of secnidazole is from about 700 milligrams to about 800 milligrams. In some embodiments, the therapeutically effective amount of secnidazole is from about 725 milligrams to about 775 milligrams. In some embodiments, the therapeutically effective amount of secnidazole is about 750 milligrams. The soft gelatin capsule compositions of the present invention may be administered at a dosage and for a duration sufficient to treat the condition sought to be treated.

In some embodiments, the excipient is a oleoyl polyoxyl-6 glyceride (e.g., Labrafil M1944 CS), mixture of medium chain triglycerides (e.g., Labrafac Lipofile WL1349), or a combination thereof. In some embodiments, the mono unsaturated fatty acid excipient is a polyoxylglyceride excipient. In some embodiments, the polyoxylglyceride excipient comprises monoglycerides Oleic acid (C18:1), diglycerides Oleic acid (C18:1), triglycerides Oleic acid (C18:1), mono-polyethylene glycol-6 esters of Oleic acid (C18:1), di-polyethylene glycol-6 esters of Oleic acid (C18:1), or any combination thereof. In some embodiments, the amount of excipient, e.g., the polyoxylglyceride excipient is from about 0.001 grams to about 3 grams. In some embodiments, the amount of excipient, e.g., the polyoxylglyceride excipient is from about 0.01 grams to about 3 grams. In some embodiments, the amount of excipient, e.g., the polyoxylglyceride excipient is from about 0.5 grams to about 3 grams. In some embodiments, the amount of excipient, e.g., the polyoxylglyceride excipient is from about 1.0 grams to about 3 grams. In some embodiments, the amount of excipient, e.g., the polyoxylglyceride excipient is from about 1 gram to about 2 grams. In some embodiments, the amount of excipient, e.g., the polyoxylglyceride excipient is from about 1 gram to about 1.5 grams. In some embodiments, the amount of excipient, e.g., the polyoxylglyceride excipient is from about 1 gram to about 1.25 grams. In some embodiments, the amount of polyoxylglyceride excipient is about 1.2 grams.

Some embodiments are directed to soft gelatin capsule compositions for vaginal administration of secnidazole comprising a soft gelatin capsule and a therapeutically effective amount of secnidazole dispersed in a mono unsaturated fatty acid excipient. In some embodiments, the therapeutically effective amount of secnidazole is from about 500 milligrams to about 1,000 milligrams. In some embodiments, the therapeutically effective amount of secnidazole is about 750 milligrams. In some embodiments, the mono unsaturated fatty acid excipient is a polyoxylglyceride excipient. In some embodiments the polyoxylglyceride excipient comprises monoglycerides Oleic acid (C18:1), diglycerides Oleic acid (C18:1), triglycerides Oleic acid (C18:1), mono-polyethylene glycol-6 esters of Oleic acid (C18:1), di-polyethylene glycol-6 esters of Oleic acid (C18:1), or any combination thereof. In some embodiments the excipient is oleoyl polyoxyl-6 glyceride (e.g., Labrafil M1944 CS), mixture of medium chain triglycerides (e.g., Labrafac Lipofile WL1349), or a combination thereof. In some embodiments, the amount of excipient, e.g., the polyoxylglyceride excipient in the composition is about 1.2 grams.

Some embodiments are directed to methods of treating bacterial vaginosis in a patient in need thereof, comprising administering intravaginally to the patient a soft gelatin capsule composition of secnidazole, comprising a therapeutically effective amount of secnidazole dispersed in a mono unsaturated fatty acid excipient. In some embodiments, the soft gelatin compositions described herein may be administered once a day for a period of 1, 2, 3, 4, 5, 6, 7 days or longer. In some embodiments, the soft gelatin compositions described herein may be administered once, twice, or three times per day for a period of 1, 2, 3, 4, 5, 6, 7 days or longer. In some embodiments, the administering the soft gel capsule compositions at the doses and frequency of administration described herein results in treatment of the bacterial vaginosis in the patient.

In some embodiments, a method of treating bacterial vaginosis in a patient in need thereof comprising administering to the patient a soft gelatin capsule composition for vaginal administration of secnidazole comprising a soft gelatin capsule, and a therapeutically effective amount of secnidazole dispersed in a mono unsaturated fatty acid excipient. In some embodiments, the therapeutically effective amount of secnidazole is from about 500 milligrams to about 1,000 milligrams. In some embodiments, the therapeutically effective amount of secnidazole is about 750 milligrams. In some embodiments, the mono unsaturated fatty acid excipient is a polyoxylglyceride excipient. In some embodiments the polyoxylglyceride excipient comprises monoglycerides Oleic acid (C18:1), diglycerides Oleic acid (C18:1), triglycerides Oleic acid (C18:1), mono-polyethylene glycol-6 esters of Oleic acid (C18:1), di-polyethylene glycol-6 esters of Oleic acid (C18:1), or any combination thereof. In some embodiments the excipient is oleoyl polyoxyl-6 glyceride (e.g., Labrafil M1944 CS), mixture of medium chain triglycerides (e.g., Labrafac Lipophile WL1349), or a combination thereof. In some embodiments, the amount of excipient, e.g., the polyoxylglyceride excipient in the composition is about 1.2 grams.

Some embodiments are directed to methods of treating a condition in a patient in need thereof, comprising administering intravaginally to the patient a soft gelatin capsule composition of secnidazole, comprising a therapeutically effective amount of secnidazole dispersed in a mono unsaturated fatty acid excipient. In some embodiments, the condition is a vaginal infection. In some embodiments, the vaginal infection is caused by an overgrowth of a bacteria such as Gardenia vaginalis. In some embodiments, the condition is an infection in the vagina caused by an anaerobic bacteria or a parasite. In some embodiments, the condition is an infection in the vagina caused by a gram negative bacteria. In some embodiments, the condition is bacterial vaginosis, trichomoniasis, or any combination thereof. In some embodiments, the condition is an imbalance of the naturally occurring bacteria in the vagina.

The soft gelatin capsule compositions described herein may be prepared, packaged, or sold in bulk, as a single unit dose or as multiple unit doses and may be administered in the conventional manner by any route where they are active.

In some embodiments, therapeutically effective amounts, daily doses, or single unit doses of the secnidazole compositions described herein may be administered once per day or multiple times per day, such as 1 to 5 doses, twice per day or three times per day. In certain embodiments, one soft gelatin capsule composition comprising a therapeutically effective amount of the secnidazole composition is administered once to said patient and the bacterial vaginosis is treated.

Embodiments are also directed to a dosage regimen for treating bacterial vaginosis in patient comprising administering secnidazole, such as secnidazole, compound to treat the conditions disclosed herein. For example, in some embodiments, the methods described herein may comprise a dosage regimen that may include a plurality of daily doses having an equal amount of secnidazole compound as the initial dose in one or more unit doses. In other embodiments, the dosage regimen may include an initial dose of secnidazole, such as secnidazole compound in one or more unit doses, then a plurality of daily doses having a lower amount of secnidazole compound as the initial dose in one or more unit doses. The dosage regimen may administer an initial dose followed by one or more maintenance doses. The plurality of doses following the administering of an initial dose may be maintenance doses.

The selection of the specific dose regimen may be adjusted or titrated by the clinician according to methods known to the clinician in order to obtain the optimal clinical response. The amount of secnidazole compound to be administered may be that amount which is therapeutically effective. The dosage to be administered may depend on the characteristics of the subject being treated, e.g., the particular animal or human subject treated, age, weight, body mass index, body surface area, health, types of concurrent treatment, if any, and frequency of treatments, and can be easily determined by one of skill in the art (e.g., by the clinician).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In all of the foregoing embodiments disclosed herein, it is to be understood that all embodiments may be further limited by using “consisting of” or “consisting essentially of” language, rather than “comprising”. When used, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### Examples

The following Examples are intended to be illustrative and are in no way intended to limit the scope of the present invention.

### Example 1—Solubility Screening for Secnidazole

This study was carried out to evaluate the solubility secnidazole in a variety of dosing vehicles according to protocol TTP-CSU-M0199. A total of 12 vehicles which were selected and agreed with the customer were used in this study.

Secnidazole Lot F130011 is a crystalline white micronized powder. The API (25-60 mg) was added to approximately 250 mg of the vehicles. After the initial addition of the API, the mixtures were shaken in a temperature-controlled vortex mixer for 24 hours at 25° C. for liquid samples and 50° C. for semi-solid samples. Additional API was added to samples where dissolution was observed after mixing for 24 hours. The mixtures were shaken for five days. Then, the suspensions were filtered using a centrifuge tube with 0.45 μm PVDF membrane filter (Millipore Durapore®). The thick filtrate was weighed into a 20-mL volumetric flask and diluted to mark with the diluent solution (50:45:5 v/v/v acetone:MeOH:water).

High-Performance Liquid Chromatography: Reverse-phase HPLC analyses were performed on a HP1200 system equipped with a G1312B binary pump, G1367C autosampler, and G1315C diode array detector. Operating HPLC parameters are shown in Table 1 below.

TABLE 1
HPLC Parameters

| Column | Zorbax SB-C18 1.8 μm, 50 × 3.0 mm | | |
|---|---|---|---|
| Diluent | IPA:MeOH:Water 50:45:5 v/v/v | | |
| Mobile Phase A | Water (with 0.05% TFA) | | |
| Mobile Phase B | Acetonitrile (with 0.05% TFA) | | |
| | Time (min) | % A | % B |
| Gradient | 0 | 100 | 0 |
| | 2.50 | 5 | 95 |
| | 2.70 | 5 | 95 |
| | 2.71 | 100 | 0 |
| | 4.00 | 100 | 0 |

| | |
|---|---|
| Flow rate | 1.5 mL/min |
| Column Temperature | 60° C. |
| Sample Temperature | 23° C. |
| Injection | 5 μL |
| Detector | UV at 290 nm |
| Analyte RT | 1.20 min |

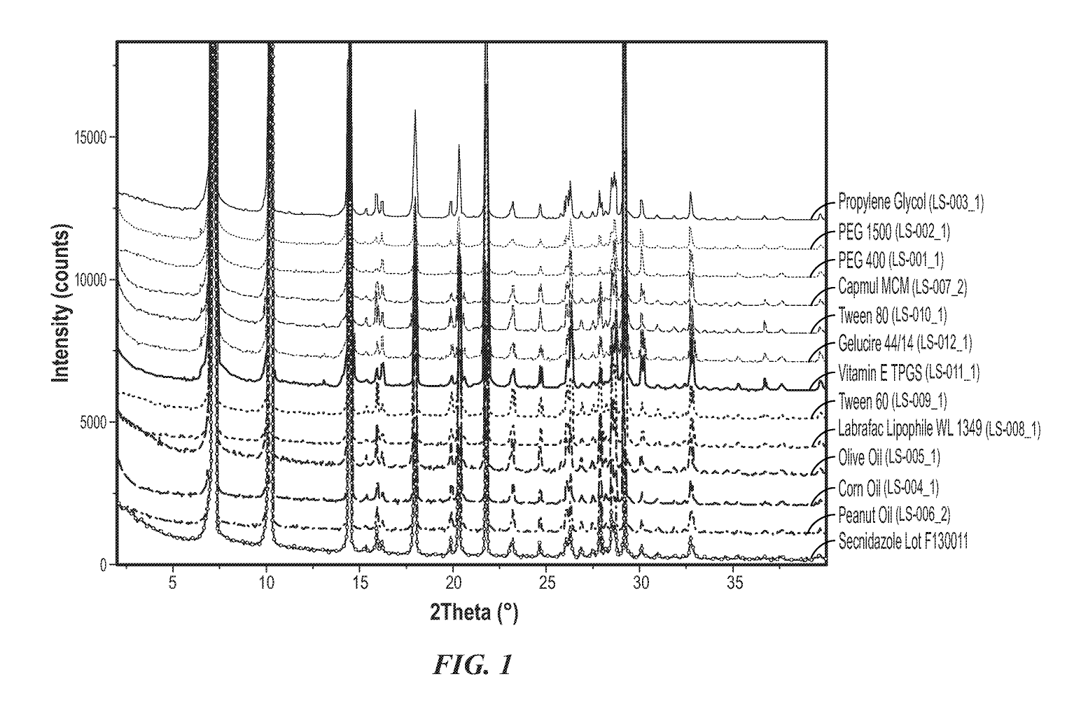

The results of both visual and HPLC solubility determinations for the supplied material are presented in Table 2 below. Secnidazole exhibited high solubility (>300 mg API/g vehicle) in propylene glycol (535 mg API/g vehicle), PEG 1500 (351 mg API/g vehicle), and PEG 400 (349 mg API/g vehicle). Moderate solubility (140-200 mg API/g vehicle) was determined in four of the 12 vehicles assessed, including Capmul MCM NF (192 mg API/g vehicle), Tween 80 (160 mg API/g vehicle), Gelucire 44/14 (151 mg API/g vehicle), and Vitamin E TPGS (140 mg API/g vehicle). Poor solubility (<23 mg API/g vehicle) was determined in the remaining five vehicles assessed, including Tween 60 (23 mg API/g vehicle), Labrafac Lipophile WL 1349 (11 mg API/g vehicle), olive oil (5 mg API/g vehicle), corn oil (4 mg API/g vehicle), and peanut oil (4 mg API/g vehicle). PXRD of the residual solids were consistent with the input form (Pattern A), as seen in FIG. 1. Select samples contained a small extraneous peak at ˜13.1° 2θ which could be a consequence of the input micronized material recrystallizing in some of the vehicles.

TABLE 2
Solubility of Secnidazole
Pattern A = Secnidazole lot F13001

| | | | Visual | | | |
|---|---|---|---|---|---|---|
| | | | Solubility | HPLC | HPLC | |
| | | Incu- | Estimate | Solubility | Solubility | Residual |
| | | bation | (mg API | (mg API | (mg API | Solids |
| | | Temp, | per g | per g | per g | Form by |
| # | Vehicle | ° C. | Vehicle) | Vehicle) | Mixture) | PXRD |
| 1 | Propylene | 25 | 510-750 | 535 | 349 | Pattern A |
| | Glycol | | | | | |
| 2 | PEG 1500 | 50 | 420-600 | 351 | 260 | Pattern A |
| 3 | PEG 400 | 25 | 230-440 | 349 | 259 | Pattern A |
| 4 | Capmul | 25 | <250 | 192 | 161 | Pattern A |
| | MCM NF | | | | | |
| 5 | Tween 80 | 25 | <230 | 160 | 138 | Pattern A |
| 6 | Gelucire | 50 | 230-400 | 151 | 131 | Pattern A |
| | 44/14 | | | | | |
| 7 | Vitamin E | 50 | 230-470 | 140 | 123 | Pattern A |
| | TPGS | | | | | |
| 8 | Tween 60 | 25 | <260 | 23 | 22 | Pattern A |
| 9 | Labrafac | 25 | <180 | 11 | 11 | Pattern A |
| | Lipophile | | | | | |
| | WL 1349 | | | | | |
| 10 | Olive Oil | 25 | <100 | 5 | 5 | Pattern A |
| 11 | Com Oil | 25 | <110 | 4 | 4 | Pattern A |
| 12 | Peanut oil | 25 | <80 | 4 | 4 | Pattern A |

### Example 2—Secnidazole Solubility and Stability

Secnidazole compositions used for further solubility screening and stability studies are shown below in Tables 3 to 5. Lot No. A00031-27 was prepared by mixing Labrafil M1944 CS, Polysorbate 80 and Lecithin and slowly adding Secnidazole to the mixture of Labrafil M1944 CS, Polysorbate 80 and Lecithin. The mixture of Secnidazole with Labrafil M1944 CS, Polysorbate 80 and Lecithin was mixed until it formed a uniform dispersion. Note, the total shown in Table 3 below may not add up to 100% due to rounding error.

TABLE 3
Lot No. A00031-27

| | Lot No. |
|---|---|
| | A00031-27 |
| Ingredients | % |
| Secnidazole | 38.9 |
| Labrafil M1944 CS | 60.2 |
| Polysorbate 80 | 0.4 |
| Lecithin | 0.4 |
| Total | 100.0% |

TABLE 4
Lot No. A00031-28B

| | Lot No. |
|---|---|
| | A00031-28B |
| Ingredients | % |
| Secnidazole | 49.8 |
| Light Mineral Oil | 49.8 |
| Lecithin | 0.5 |
| Total | 100.0% |

Lot No. A00031-28B was prepared by mixing mineral oil with Lecithin and slowly adding Secnidazole to the mixture of mineral oil and Lecithin and mixing till a uniform dispersion was formed. The composition is shown in Table 4 below (total may not add up to 100% due to rounding error).

Lot Nos. A00031-29 and A00031-31B were formed by mixing Polyethylene Glycol 400, Propylene Glycol and Polyethylene Glycol 400, heating the mixture at 45-60° C. along with mixing until a clear solution is obtained. Secnidazole was added to the clear solution with mixing until a clear solution was obtained. Purified water was added as the last step with mixing. The composition is shown in Table 5 below.

TABLE 5
Lot No. A00031-29 and A00031-31B

| | Lot No. | Lot No. |
|---|---|---|
| | A00031-29 | A00031-31B |
| Ingredients | % | % |
| Secnidazole | 23.4 | 21.1 |
| Polyethylene Glycol 400 | 67.6 | 60.8 |
| Propylene Glycol | 5.0 | 4.5 |
| Polyethylene Glycol 4600 | 4.0 | 3.6 |
| DI Water | 0.0 | 10.0 |
| Total | 100.0% | 100.0% |

Table 6 below shows the solubility of Secnidazole Lot Nos. A00031-27, A00031-28B, A00031-29, and A00031-31B.

TABLE 6
Secnidazole Solubility

| | Description | Solubility |
|---|---|---|
| Lot No. | (Fill solutions saturated with API) | mg/g |
| A00031-27 | Labrafil M1944/Lecithin/Polysorbate 80 | 26.2 |
| A00031-28B | Mineral Oil Lecithin | 0.2 |
| A00031-29 | PEG 400/PG/PEG 4600 | 364.9 |
| A00031-31B | PEG 400/PG/PEG 4600 10% water | 281.1 |

Secnidazole was found to have very low solubility in Labrafil M1944 and mineral oil-based composition. Higher solubility was observed in the PEG 400/PG/PEG 4600 system and the presence of water appeared to lower solubility in the PEG 400/PG/PEG 4600 hydrophilic system. Table 7 below shows the stability of Secnidazole formulations at 40° C.

TABLE 7
Stability of Secnidazole Formulations at 40° C.

| | | Initial | | | 2 Weeks at 40° C. | | | 4 Weeks at 40° C. | | | 12 Weeks at 40° C. | | |
|---|---|---|---|---|---|---|---|---|---|---|---|---|---|
| | | Assay | Impurities | | Assay | Impurities | | Assay | Impurities | | Assay | Impurities | |
| Lot No. | Description | mg/g | RRT | % | mg/g | RRT | % | mg/g | RRT | % | mg/g | RRT | % |
| A00031-27 | Secnidazole | 394.4 | RRT 0.53 | <0.05 | 392.4 | RRT 0.53 | <0.05 | 446.9 | RRT 0.54 | <0.05 | 392.3 | RRT 0.53 | <0.05 |
| | Suspension | | RRT 0.65 | n/a | | RRT 0.65 | n/a | | RRT 0.65 | <0.05 | | RRT 0.65 | n/a |
| | in Labrafil | | RRT 0.80 | <0.05 | | RRT 0.81 | <0.05 | | RRT 0.82 | <0.05 | | RRT 0.81 | <0.05 |
| | M1944 (38.9%) | | RRT 1.15 | 0.48 | | RRT 1.15 | 0.48 | | RRT 1.15 | 0.48 | | RRT 1.15 | 0.48 |
| A00031-28B | Secnidazole | 498.8 | RRT 0.53 | <0.05 | 510.7 | RRT 0.53 | <0.05 | 504.5 | RRT 0.54 | <0.05 | 503.9 | RRT 0.53 | <0.05 |
| | Suspension | | RRT 0.64 | <0.05 | | RRT 0.64 | n/a | | RRT 0.66 | <0.05 | | RRT 0.65 | n/a |
| | in Mineral | | RRT 0.81 | <0.05 | | RRT 0.81 | <0.05 | | RRT 0.82 | <0.05 | | RRT 0.81 | <0.05 |
| | Oil (49.8%) | | RRT 1.15 | 0.48 | | RRT 1.15 | 0.48 | | RRT 1.15 | 0.48 | | RRT 1.15 | 0.48 |
| A00031-29 | Secnidazole | 236.3 | RRT 0.53 | <0.05 | 235.4 | RRT 0.53 | <0.05 | 236.1 | RRT 0.53 | <0.05 | 235.2 | RRT 0.53 | <0.05 |
| | Solution | | RRT 0.65 | <0.05 | | RRT 0.65 | n/a | | RRT 0.66 | n/a | | RRT 0.65 | n/a |
| | without | | RRT 0.80 | <0.05 | | RRT 0.81 | <0.05 | | RRT 0.82 | <0.05 | | RRT 0.82 | <0.05 |
| | Water (23.4%) | | RRT 1.15 | 0.48 | | RRT 1.15 | 0.47 | | RRT 1.15 | 0.48 | | RRT 1.15 | 0.48 |
| A00031-31B | Secnidazole | 215.9 | RRT 0.53 | <0.05 | 210.5 | RRT 0.53 | <0.05 | 213.2 | RRT 0.53 | <0.05 | 212.2 | RRT 0.53 | <0.05 |
| | Solution | | RRT 0.81 | <0.05 | | RRT 0.81 | <0.05 | | RRT 0.82 | <0.05 | | RRT 0.81 | <0.05 |
| | without | | RRT 1.15 | 0.47 | | RRT 1.15 | 0.48 | | RRT 1.15 | 0.48 | | RRT 1.15 | 0.48 |
| | Water (20.9%) | | | | | | | | | | | | |

Assay values in agreement with theoretical amount of drug in formulations. All formulations stable with no significant changes in assay or impurities levels after 12 weeks at 40° C.

Although exemplary embodiments have been disclosed, it will be apparent to those skilled in the art that various changes and modifications can be made which will achieve some of the advantages of embodiments without departing from the spirit and scope of the disclosure. Such modifications are intended to be covered by the appended claims in which the reference signs shall not be construed as limiting the scope.

The above description of illustrated embodiments, including what is described in the Abstract, is not intended to be exhaustive or to limit the embodiments to the precise forms disclosed. Although specific embodiments and examples are described herein for illustrative purposes, various equivalent modifications can be made without departing from the spirit and scope of the disclosure, as will be recognized by those skilled in relevant art.

The various embodiments described above can be combined to provide further embodiments. Aspects of the embodiments can be modified, if necessary to employ concepts of the various references and/or products referred to in this application to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to Which such claims are entitled. Accordingly, the claims are not limited by the disclosure.

What is claimed is:

1. A soft gelatin capsule composition for vaginal administration of secnidazole comprising a soft gelatin capsule and a therapeutically effective amount of secnidazole dispersed in a mono unsaturated fatty acid excipient.

2. The soft gelatin capsule composition of claim 1, wherein the therapeutically effective amount of secnidazole is from about 500 milligrams to about 1,000 milligrams.

3. The soft gelatin capsule composition of claim 1, wherein the therapeutically effective amount of secnidazole is about 750 milligrams.

4. The soft gelatin capsule composition of claim 1, wherein the mono unsaturated fatty acid excipient is a polyoxylglyceride excipient.

5. The soft gelatin capsule composition of claim 4, wherein the polyoxylglyceride excipient comprises monoglycerides Oleic acid (C18:1), diglycerides Oleic acid (C18:1), triglycerides Oleic acid (C18:1), mono-polyethylene glycol-6 esters of Oleic acid (C18:1), di-polyethylene glycol-6 esters of Oleic acid (C18:1), or any combination thereof.

6. The soft gelatin capsule composition of claim 1, wherein the mono unsaturated fatty acid excipient is Labrafil M1944 CS, Labrafac Lipophile WL1349, or a combination thereof.

7. The soft gelatin capsule composition of claim 4, wherein the amount of polyoxylglyceride excipient is about 1.2 grams.

8. The soft gelatin capsule composition of claim 6, wherein the amount of the mono unsaturated fatty acid excipient is about 1.2 grams.

9. A method of treating bacterial vaginosis in a patient in need thereof comprising:
administering a soft gelatin capsule composition to the patient wherein the capsule composition comprises a soft gelatin capsule and a therapeutically effective amount of secnidazole dispersed in a mono unsaturated fatty acid excipient and the capsule composition is administered intravaginally to the patient.

10. The method of claim 9, wherein the soft gelatin capsule composition is administered intravaginally to the patient and said bacterial vaginosis is treated.

11. The method of claim 9, wherein the therapeutically effective amount of secnidazole is from about 500 milligrams to about 1,000 milligrams.

12. The method of claim 9, wherein the therapeutically effective amount of secnidazole is about 750 milligrams.

13. The method of claim 9, wherein the mono unsaturated fatty acid excipient is a polyoxylglyceride excipient.

14. The method of claim 13, wherein the polyoxylglyceride excipient comprises monoglycerides Oleic acid (C18:1), diglycerides Oleic acid (C18:1), triglycerides Oleic acid (C18:1), mono-polyethylene glycol-6 esters of Oleic acid (C18:1), di-polyethylene glycol-6 esters of Oleic acid (C18:1), or any combination thereof.

15. The method of claim 9, wherein the mono unsaturated fatty acid excipient is Labrafil M1944 CS, Labrafac Lipophile WL1349, or a combination thereof.

16. The method of claim 13, wherein the amount of polyoxylglyceride excipient is about 1.2 grams.

17. The method of claim 15, wherein the amount of the mono unsaturated fatty acid excipient is about 1.2 grams.
